(19) United States
(12) Plant Patent Application Publication
(10) Pub. No.: US 2015/0020272 P1
(43) Pub. Date: Jan. 15, 2015

(54) Sea oats named 'La12-202'

(50) ;

(71) Applicant: Board of Supervisors of Louisiana State University and Agricultural and Mechanical College, Baton Rouge, LA (US)

(72) Inventors: Carrie Ann Knott, Baton Rouge, LA (US); Michael David Materne, Baton Rouge, LA (US); Prasanta Kumar Subudhi, Baton Rouge, LA (US); Niranjan Baisakh, Baton Rouge, LA (US); Herry Sutjahjo Utomo, Rayne, LA (US); Stephen Alan Harrison, Baton Rouge, LA (US)

(21) Appl. No.: 13/987,224

(22) Filed: Jul. 12, 2013

(51) Int. Cl. A01H 5/00

(52) U.S. Cl. PLT/387

(57) ABSTRACT

A new variety of sea oats identified as ‘LA12-202’ is disclosed as being genetically different from ‘Caminada’, and as being tall; having a vigorous growth habit; high stem density; large stem diameter and leaf width; large spikelets with numerous florets per spikelet; many panicles per plant; and flowering in early- to mid-July in natural beach environments.

The development of this invention was partially funded with Hatch Act Formula Funds (Project number LAB93864) and through grants from the United States Department of Agriculture's National Institute of Food and Agriculture (Agreement numbers: 2010-34396-21191, 2009-34396-20051, 2008-34396-19316, 2006-34396-17624, 2005-34396-16498).

The Government may have certain rights in this invention.

This invention pertains to a new and distinct variety of sea oats.

### BACKGROUND OF THE INVENTION

Sea oats (Uniola paniculata L.) is a perennial rhizomatous grass native to beaches along the Atlantic and Gulf of Mexico coasts in the United States. It is used in numerous beach restoration projects to collect blowing sand and stabilize sand dunes, thus decreasing coastal erosion. In Louisiana, one (1) source of sea oats, identified as ‘Caminada’ germplasm, is used extensively. The widespread use of a single germplasm source can result in inferior plants being used in restoration projects and reduce genetic variation. Inferior plants may not survive as well or produce as vigorous or large plants as improved varieties. Reduced genetic variation can reduce the ability of plants to adapt to environmental changes. ‘LA12-202’, along with ‘LA12-201’ and ‘LA12-203’, was invented to provide improved, genetically diverse sea oats varieties for northern Gulf of Mexico beach restoration projects.

Sea oats seeds were collected from fifty-eight (58) sea oats populations throughout the Atlantic and northern Gulf of Mexico coasts in 2001. Preliminary work, which preceded this invention, evaluated sea oats genetic diversity of a random sample of plants produced from the collected seeds (Parami, 2003. http://ehis.ebscohost.com/eds/detail?vid=2&sid=8ecded68-fed4-4583-a5f3-93985948a0fa%40sessionmgr4&hid=7&bdata=JnNpdGU9ZWRzLWxpdmUmc2NvcGU9c210Z Q%3d%3d#db=cat00252a&AN=lalu.2119724 and Subudhi et al., 2005. Theor. Appl. Genet. 111:1632). In the preliminary work, no efforts were made to asexually reproduce ‘LA12-202’ for any purpose other than to provide plant material for plant performance evaluations. The new sea oats varieties ('LA12-201′, ‘LA12-202’ and ‘LA12-203’) were designated as varieties and reproduced asexually beginning in 2010.

### BRIEF SUMMARY OF INVENTION

### Genus and Species Name

‘LA12-202’ is a new sea oats (Uniola paniculata L.) variety that is vigorous, has high stem density, large spikelets with numerous florets per spikelet, and produces many panicles. ‘LA12-202’ is genetically different from ‘Caminada’ and two (2) newly developed sea oats varieties, ‘LA12-201’ and LA12-203’. Genetic diversity was determined prior to variety invention using five hundred thirty four (534) Amplified Fragment Length Polymorphism (AFLP) loci (Bertrand-Garcia et al., Euphytica 185:103). ‘LA12-202’ is recommended for beach restoration projects in the northern Gulf of Mexico, especially Louisiana, where vigorous and genetically different sea oats varieties that survive and perform well before and after major hurricane events are desired.

### Variety Denomination

This new and distinct sea oats variety, identified as ‘LA12-202’, is characterized by its vigorous growth habit, dark green foliage, high stem density, large spikelets, and numerous panicles that flower earlier than ‘Caminada’ in natural beach environments.

### BRIEF DESCRIPTION OF THE DRAWINGS

The file of this Patent contains at least one photograph executed in color. Copies of this Patent or Patent Application with color drawings(s) will be provided by the US Patent and Trademark Office upon request and payment of the necessary fees.

FIG. 1 is a color photograph of the novel sea oats variety identified as ‘LA 12-202’.

FIG. 2 is a color photograph of sea oats germplasm identified as ‘Caminada’.

### DETAILED BOTANICAL DESCRIPTION

‘LA12-202’ was developed from seed collected from sea oats plants growing on Santa Rosa Island, Fla., (30° 23′ 43.2″ N; 86° 41′ 57.8″ W) in September of 2001. Collected seeds were germinated in a greenhouse at Baton Rouge, La. in 2003. Sea oats seedlings were transplanted to beach locations in Louisiana and Mississippi to evaluate survival and performance in 2003. In 2005, genotypes were selected for acceptable performance at Holly Beach, La., and Long Beach, Miss. Stem and rhizome material was harvested and planted into containers in Baton Rouge, La. This asexual reproduction was used solely to multiply experimental lines for evaluation in replicated trials and not for the asexual reproduction of the variety. Replicated trials were initiated in 2008.

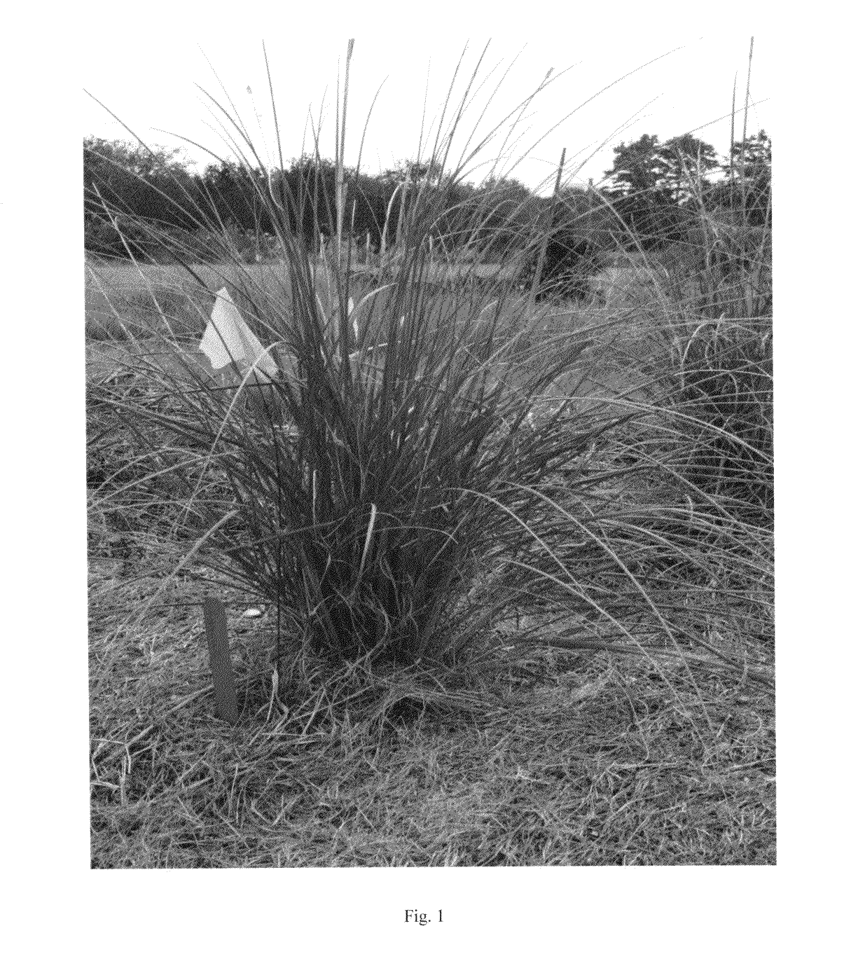

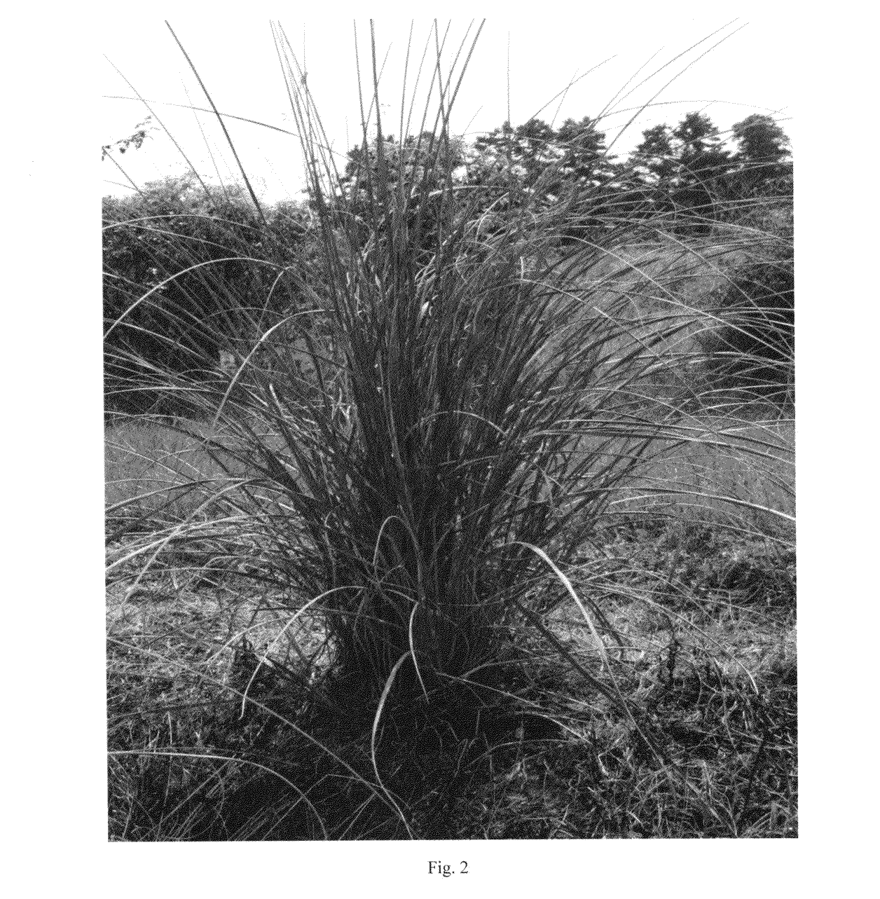

In general, ‘LA12-202’ leaves are darker green [7.5 GY (5/4)] than ‘Caminada’ [5 GY (5/4)] when color is determined with the MUNSELL®. Book of Color (Munsell Color, Gretag Macbeth LLC, 617 Little Britain Road, New Windsor, N.Y. 12553-6148) (FIG. 1 and FIG. 2). ‘LA12-202’ is taller and produces more panicles (Table 1) and has similar stem diameters, leaf widths, number of spikelets per panicle, and florets per spikelet as ‘Caminada’ (Table 1). In beach environments, ‘LA12-202’ flowers early to mid-July, while ‘Caminada’ flowers mid- to late July. ‘LA12-202’ is genetically identical and stable when produced from rhizome material; however variations in plant appearance can be caused by production conditions and does not reflect genetic differences.

TABLE 1

| | | | | | | | Spikelets | | Florets | | Number | |
|---|---|---|---|---|---|---|---|---|---|---|---|---|
| | | | Stem | | Leaf | | per | | per | | of | |
| Variety | Height (cm) | | Diameter (mm) | | Width (mm) | | Panicle | | Spikelet | | Panicles | |
| ‘LA12-202’ | 53.6 | a^{†} | 5.4 | b | 8.0 | ab | 278.3 | a | 6.3 | ab | 6.5 | a |
| ‘Caminada’ | 41.7 | b | 5.2 | b | 7.0 | b | 257.4 | ab | 5.3 | b | 2.5 | b |
| ‘LA12-201’ | 47.8 | ab | 8.0 | a | 9.0 | a | 222.0 | b | 7.9 | a | 7.5 | a |

^{†}Means within the same column that are followed by different letters are significantly different (t test, p < 0.10).

‘LA12-202’ and ninety-five (95) additional genotypes were evaluated in replicated beach trials at Gulfport, Miss., from 2008-2009. In 2010, ‘LA12-202’ was identified as a superior cultivar. The variety ‘LA12-202’ is asexually propagated in Baton Rouge, La.

### Explanation of Tests Conducted

Un-Replicated Beach Trials: In 2003, a total of 1098 sea oats seedlings were transplanted to Holly Beach (Cameron Parish), La., in un-replicated preliminary trials. In 2004, a total of 902 seedlings were transplanted to Long Beach (Harrison County), Miss., in un-replicated preliminary trials. Subjective evaluations were measured twice, once per year from 2004 to 2005, for plant vigor, tillering capacity and spread. The subjective evaluations were completed with a 0 to 10 scale, where 0 indicated a plant that was dead, had no tillers, or did not spread from original transplant location, and 10 indicated a plant that was extremely vigorous, had numerous tillers, or spread far from the original transplant location. In 2005, genotypes were selected and stem and rhizome material was harvested and planted into containers at Baton Rouge, La. This asexual reproduction was used solely to multiply experimental lines for evaluation in replicated trials and not for the asexual reproduction of the variety.

Replicated Beach Trials:

In 2008, a total of 96 clones were transplanted to Gulfport (Harrison County), Miss., and evaluated from 2008 to 2009. Plant vigor was evaluated a total of 9 times: 3 months after transplant (15 Aug. 2008); 2 and 3 weeks after Hurricane Gustav made landfall (17 Sep. and 24 Sep. 2008); 11, 12, 13, 14, and 15 months after transplant (9 Apr., 11 May, 3 June, 13 July, 4 Aug. 2009); and 1 year after Hurricane Gustav made landfall (1 Sep. 2009). Stem density was measured as the number of stems per 100 cm² of beach area in 2009 on 9 April, 11 May, 3 June, 13 July, 4 August, and 1 September. Plant height was measured from the soil surface to the tallest point of the leaf canopy (cm) in 2009 on 9 April, 11 May, 3 June, 13 July, 4 August, and 1 September. In 2009, the number of spikelets per panicle was measured, and the number of florets per spikelet was determined by averaging the number of florets for 10 random spikelets per panicle.

In 2009, the genetic similarity of ‘LA12-202’, ‘Caminada’, and 76 additional genotypes was determined using 534 Amplified Fragment Length Polymorphisms (AFLP) (Bertrand-Garcia et al., 2012. Euphytica 185:103; Knott et al., 2012. J. Plant Registrations 6:289). Jaccard similarity coefficients were calculated to determine genetic similarity of the 534 AFLP bands as al(a+b+c) where a=the number of matching present bands (“1” and “1”), b=the number of unmatched bands (“1” and “0”), and c=the number of unmatched bands (“0” and “1”) (Subudhi et al., 2005. Euphytica 111:1632-1641).

### EXAMPLE 1

### Un-Replicated Beach Trials

In un-replicated beach trials at Long Beach, Miss. (described in Paragraph 0013), the mean vigor and plant spread was numerically larger for ‘LA12-202’ than the average of all genotypes tested, while the panicle size and number for ‘LA12-202’ was numerically smaller than the average of all genotypes tested (Table 2).

TABLE 2

| | | Plant | Panicle Size |
|---|---|---|---|
| Variety | Vigor^{†} | Spread^{§} | and Number^{¶} |
| ‘LA12-202’ | 6.0 | 6.0 | 3.5 |
| Average | 4.7 | 5.2 | 3.9 |

^{†}Vigor was visually estimated on a scale of 0-10 (0 = dead; 10 = excellent).
^{§}Plant spread was visually estimated on a scale of 0-10 (0 = no plant spread; 10 = plant spread far from original plant).
^{¶}Panicle size and number was visually estimated on a scale of 0-10 (0 = no panicles; 10 = numerous large panicles).

### EXAMPLE 2

### Replicated Beach Trials

In replicated beach trials at Gulfport, Miss., ‘LA12-202’ (described in Paragraph 0014) was more vigorous, taller, and had a higher stem density than ‘Caminada’ (Table 3).

TABLE 3

| | | | Height | | Number of Stems | |
|---|---|---|---|---|---|---|
| Variety | Vigor^{†} | | (cm) | | per 100 cm² | |
| ‘LA12-202’ | 5.2 | al^{‡} | 53.6 | a | 3.8 | a |
| ‘Caminada’ | 3.7 | b | 41.7 | b | 2.7 | b |
| ‘LA12-201’ | 5.1 | a | 47.8 | ab | 3.9 | a |

^{†}Vigor was visually estimated on a scale of 0-10 (0 = dead; 10 = excellent).
^{‡}Means within the same column that are followed by different letters are significantly different (t test, p < 0.10).

### EXAMPLE 3

### Genetic Similarity Analysis

Genetic similarity of ‘LA12-202’, ‘Caminada’, and ‘LA12-201’ was determined with Jaccard similarity coefficients from 534 Amplified Fragment Length Polymorphisms (AFLP). ‘LA12-202’ and ‘LA12-201’ were the least similar (0.9105) while ‘LA12-201’ and ‘Caminada’ were most similar (0.9258) (Table 4).

TABLE 4

| Variety | ‘LA12-202’ | ‘Caminada’ | ‘LA12-201’ |
|---|---|---|---|
| ‘LA12-202’ | 1.0000 | | |
| ‘Caminada’ | 0.9156 | 1.0000 | |
| ‘LA12-201’ | 0.9105 | 0.9258 | 1.0000 |

We claim:

1. A new and distinct variety of Uniola paniculata named ‘LA12-202’ as described and illustrated in the specification herein.
